(19) United States
(12) Patent Application Publication
(10) Pub. No.: US 2007/0123780 A1
(43) Pub. Date: May 31, 2007

(54) Low-frequency focus ultrasound-generating device for tumor treatment using ultrasound irradiation microbubble agent

(76) Inventor: Wei Wu, Nanjing (CN)

Correspondence Address: TRASK BRITT, P.O. BOX 2550, SALT LAKE CITY, UT, 84110, US

(21) Appl. No.: 11/492,452

(22) Filed: Jul. 25, 2006

(30) Foreign Application Priority Data
Nov. 29, 2005 (CN) 200510122608.2

(51) Int. Cl. A61B 8/00

(52) U.S. Cl. 600/439

(57) ABSTRACT

The invention relates to a low-frequency focus ultrasound-generating device and its application in clinical physical therapy to cause embolism of new blood vessels of tumor. The device comprises a hand grip, an ultrasound output, a half-ellipsoid reflector, and a water sack. The half-ellipsoid reflector is disposed on the forward end of the hand grip. The rear part of the ultrasound output is set in the hand grip, while the front part of the ultrasound output is set in the half-ellipsoid reflector. On a lateral side of the ultrasound output in the front of the hand grip, there is a tie-in for water infusion and gas exhaust. The inner portion of the tie-in is in communication with the inner portion of the half-ellipsoid reflector. At an opening on the forward end of the half-ellipsoid reflector is disposed the water sack. The front face of said ultrasound output lies in a focus of the half-ellipsoid reflector.

### TECHNICAL FIELD

The invention relates to noninvasive tumor therapy, more particularly, to a low-frequency focus ultrasound-generating device for tumor treatment using ultrasound irradiation microbubble agent, which device is applied in embolism treatment of tumors.

### BACKGROUND

Cancer has been one of the most serious diseases of humankind. So far, fundamental cancer treatment methods include surgery, actinotheraphy, chemotheraphy and immunotherapy. However, these methods are not satisfactory for the treatment of the disease. Moreover, actinotheraphy and chemotheraphy tend to cause side effects. How to control tumor growth, and/or how to cure malignant tumors safely and efficiently, became an urgent issue.

In recent years, research on the application of microbubble agents in ultrasonic imaging diagnosis has become popular. However, the research on how to utilize microbubble agents and the like to enhance the effects of ultrasound for clinic treatment is still in an early stage. Due to the small amount of cavitation nucleus in human body, an additional microbubble agent is needed to increase the amount of cavitation nucleus during treatment using ultrasonic cavitation effects.

Most of current ultrasonic therapies for tumors are based on the heat effect of ultrasound with high frequency and power. The heat generated by the high-frequency ultrasound is focused on a local part of the tumor, thus destroying the tumor organization. Due to the high power of the ultrasound and the difficulty of controlling cavitation effects, high frequency ultrasound may cause undesired effects to normal tissues and organs, and therefore it is not suitable for embolism treatment of tumors.

Research on ultrasonic cavitation indicates that if there is a cavitation nucleus in a liquid and if the nucleus contains vapor, the intensity of the liquid can be calculated by the following formula:

$${p}_{t} = - {p}_{p} + \frac{2}{3 \sqrt{3}} \sqrt{\frac{{( \frac{2 σ}{{R}_{0}} )}^{3}}{{p}_{0} - {p}_{p} + \frac{2 σ}{{R}_{0}}}} \qquad ( 1 )$$

Wherein pₚ denotes vapor pressure, R₀ denotes original radius of cavitation nucleus, σ denotes surface tension, and p₀ denotes static pressure of the liquid.

Formula (1) indicates that the increase of radius R₀ results in a decrease of liquid intensity, and vice versa. The liquid intensity is relatively weak at or about cavitation nucleus, where cavitation starts.

The smallest pressure for generating cavitation is designated as the threshold of liquid cavitation. If the static pressure is p₀, and the alternation sound pressure of sound wave is pₘ, then the pressure in the liquid is changed as p₀±pₘ. If pₘ>p₀, then minus pressure is formed to make the cavitation nucleus inflate; and when p₀−pₘ>p*, cavitation occurs. If the radius is equivalent to R₀, the threshold of ultrasonic cavitation is:
p_{c}=p₀+pₜ (2)

To substitute formula (1) into (2), the threshold of ultrasonic cavitation is:

$${p}_{c} = {p}_{0} - {p}_{p} + \frac{2}{3 \sqrt{3}} \sqrt{\frac{{( \frac{2 σ}{{R}_{0}} )}^{3}}{{p}_{0} - {p}_{p} + \frac{2 σ}{{R}_{0}}}} \qquad ( 3 )$$

It is to be understood that the threshold of ultrasonic cavitation alters with different kinds of liquid. For the same liquid, it alters with change of temperature, pressure, amount of vapor, as well as size and distribution of cavitation nucleus. The cavitation threshold of water p_{c} goes up as the vapor amount (relative saturation vapor amount) of water goes down. The quantity of cavitation nucleus is reduced due to the increase of static pressure and decrease of the amount of vapor, thereby the cavitation threshold is increased. Experiments have proven that when the amount of vapor in a liquid is large, static pressure has greater influence on the threshold; and when the amount of vapor in a liquid is small, static pressure has smaller influence on the threshold. Cavitation threshold is also associated with liquid viscosity. In a liquid of high viscosity, cavitation threshold becomes a little higher. The threshold also depends on how long sound wave is applied. Cavitation threshold decreases with the increase of the duration of time for which the sound wave is applied, especially in water with air therein. Cavitation threshold is also proportional to sound frequency. The higher the sound frequency is, the higher the cavitation threshold. In addition, ultrasonic cavitation is influenced by the bubbles' dynamic process, the growth and closure of cavitation bubbles and other factors.

Literature indicates that the ultrasonic pressure or intensity required by the cavitation effect generated on the operating frequency of tens of kHz is only one tenth or one ten thousandth of that of high-frequency ultrasound (i.e., several hundreds to thousands KHz). In addition, the amount of gas in a liquid is an important factor which has influence on ultrasonic cavitation generation.

As shown in FIG. 1, the ultrasonic power required by cavitation generation on operating frequency of tens of kHz in water with air (when microbubbles exist) is in the order of 10⁻¹ W/cm². Accordingly, we selected ultrasonic output powers of 0.5 W, 1.0 W, and 1.5 W, in combination with ultrasonic microbubble agent, in ultrasonic biological effect (safety) tests. The results showed that in the combination experiment of ultrasound with microbubble agent in tissue, when the frequency is higher (1.5 W), ALT of animal blood rises; when it is lower (1 W), there is no abnormal alternation of the function of liver and kidney; and when it is decreased to 0.5 W, there is nothing abnormal in liver and kidney as well as no significant damages in liver, kidney, endocardium, brain, skin and subcutaneous tissue. Blood gas analysis indicates that in 10 to 60 minutes after an equivalent dosage of microbubble agent is injected, CO₂ pressure in blood gas does not have a significant change.

When animals suffering from tumors are treated by ultrasound in combination with a microbubble agent at the output frequency of 0.5 W for 30 seconds, blood vessel embolism and tumor necrosis are found in and around the tumor, but normal tissues are not damaged and no blood vessel embolism are found in the normal tissues.

According to literature, with regard to the pressure intensity (sound intensity) generated under ultrasound by cavitation nucleus (i.e., gas nucleus or microbubble), the pressure intensity P(r) generated at the outer surface, which is r from the center of the bubble when the bubble is closed, is denoted as follows:

$$p ( r ) = {p}_{0} + R [ {Z}^{′} Q \frac{3 r - 4}{r - 1} + \frac{Z Q}{r - 1} + {P}_{O} ( Z - 4 ) ] - \frac{{R}^{4}}{3 {r}^{4}} [ {P}_{O} ( Z - 1 ) - Q \frac{{Z}^{′} - Z}{r - 1} ] \qquad ( 4 )$$

It is evident that the pressure intensity becomes the biggest when the bubble was pressed with the smallest radius. The curve in FIG. 2 is drawn according to formula (4), which displays the intensity of stimulated wave while the bubble is closed. If the original radius is larger, then the closed radius is smaller (Z is larger), and thus the amplitude of stimulated wave becomes larger. Actually, Rm (the largest radius of bubble) is dependent on ultrasonic frequency and amplitude Pm of ultrasonic pressure. Rm is increased as Pm is increased, and therefore stimulated wave generated becomes stronger. When ultrasonic frequency is low, the active time period is longer, and therefore the bubble closes after it swells to a considerable degree.

Therefore, blood vessel embolism in a tumor can be generated by lower ultrasonic intensity (power) using low-frequency focus ultrasound irradiation of microbubble agent, so that the channel for blood and nutrition supply for tumor is blocked. Hence, the object to control tumor growth, treat tumors, and avoid undesired damage to normal tissue and organs can be achieved.

### SUMMARY OF INVENTION

The present invention provides a low-frequency focus ultrasound-generating device for tumor treatment used with ultrasound irradiation microbubble agent. Application of the device can control tumor growth, treat tumors and avoid undesired damages to normal tissues and organs.

The device of the invention comprises a hand grip, an ultrasound output, a half-ellipsoid reflector, and a water sack. The half-ellipsoid reflector is disposed on the forward end of the hand grip. The rear part of the ultrasound output is set in the hand grip, while the front part of the ultrasound output is set in the half-ellipsoid reflector. On a lateral side of the ultrasound output in the front of the hand grip, is a tie-in for water infusion and gas exhaust. The inner portion of the tie-in is in communication with the inner portion of the half-ellipsoid reflector. At an opening on the forward end of the half-ellipsoid reflector is disposed the water sack.

In one embodiment of the device, the front face of the ultrasound output lies in a focus of the half-ellipsoid reflector.

In one embodiment of the device, the water sack is fixed at the opening on the forward end of the half-ellipsoid reflector by a fixture.

The low-frequency focus ultrasonic device produces low-frequency focus ultrasound which irradiates the microbubble agent through the skin, which microbubble agent is injected through the peripheral vein in the local areas of a tumor under the guide of an ultrasonic diagnostic instrument or any other medical imaging system, and helps to form embolism in new vessels of the tumor and block the channels for blood and nutrition supply for the tumor, so that the purpose of tumor growth control and treatment is achieved. Based on the principle of acoustics, when ultrasounds are irradiated from one focus (F1) of an ellipsoid defined by formula (5), they may gather at the other focus (F2).

$$\frac{{x}^{2}}{{a}^{2}} + \frac{{y}^{2}}{{b}^{2}} = {c}^{2} \qquad ( 5 )$$

When the half-ellipsoid is used to irradiate ultrasound from outside of a patient body, a corresponding ultrasonic power (sound intensity) can be attained, thus inducing cavitation effects of the microbubble agent injected through the peripheral vein of the human body. The cavitation effect of the microbubble agent will destroy and/or block new blood vessels in a tumor, and block channels for blood and nutrition supplies for the tumor. Therefore, tumors can be treated and/or controlled without undesired effects to normal tissues and organs.

In recent years, research on the application of microbubble agents in ultrasonic imaging diagnosis has become popular; however, the research on how to utilize microbubble agents and the like to enhance the biological effect of ultrasound for clinic treatment of tumors is still in an early stage around the world.

Due to the small amount of cavitation nucleus in human body, an additional microbubble agent is needed to increase the amount of cavitation nucleus during the treatment using ultrasonic cavitation effect.

Most of current ultrasonic therapies for tumors are based on the heat effect of ultrasound with high frequency and power. The heat generated by high-frequency ultrasound focused on a local part of the tumor thus destroys the tumor organization. Due to the high power of the ultrasound and the difficulty to control the cavitation effects, high frequency ultrasound may cause undesired harms to normal tissue and organs, and therefore it is not suitable for embolism treatment of tumors.

Studies indicate that in tissues containing a microbubble agent, a low dosage of ultrasounds can induce the sound-hole effect, which effect used to be induced by a high dosage of pure power ultrasounds. The low dosage ultrasound method has been utilized in facilitating ablation of thrombus.

In one experiment, a microbubble agent, which is usually used in ultrasonic diagnosis, was used with low frequency ultrasound irradiation. The microbubble agent can reach tissue and organs with blood flow to increase the level of cavitation nucleus in local tissues. In the experiment, low power ultrasound irradiation was applied to realize cavitation of microbubbles, thus causing fragmentation of micro-vessel walls and some adjacent tissues, activation of intrinsic and extrinsic coagulation, and induction of large area embolism in capillary vessels. Induction of large area embolism in blood vessels results in blockage of direct blood supply in the tumor region. It has been shown, in the region containing no microbubbles, little embolism was caused. Ultrasonic irradiation alone induced a low ratio of embolism, and only 34.15% vessels were embolized to some degree. However, when ultrasonic irradiation and microbubble agent were combined in use, the ratio went up to 89.11%.

In previous research, the inventor used non-focus low frequency and power ultrasonic irradiation device which has low target location and can hardly control effective treatment region. The new focus device has been improved to gain higher efficiency of treatment while avoiding negative side effects.

### DESCRIPTION OF THE FIGURES

FIG. 1 is a plot indicating that the threshold sound intensity of cavitation varies with ultrasonic frequency.

FIG. 2 is a plot indicating the pressure amplitude of a stimulated wave generated when bubbles are closing.

FIG. 3 is a structural schematic view of the present device, showing: a spring 1, a hand grip 2, a cable 3, a plug 4, an ultrasound output 5, a tie-in 6, a half-ellipsoid reflector 7, a fixture 8, a screw 9, and a water sack 10.

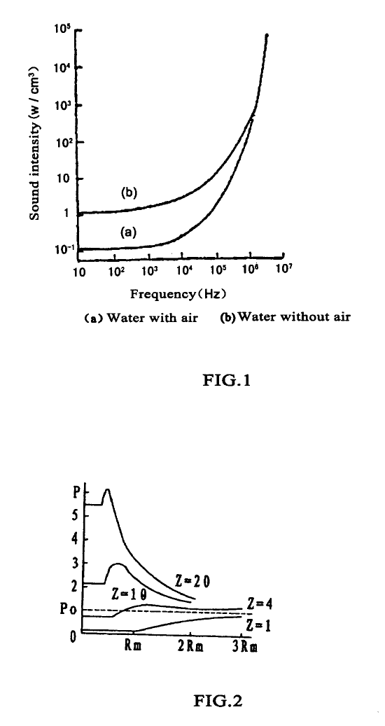

### DETAILED DESCRIPTION OF THE INVENTION

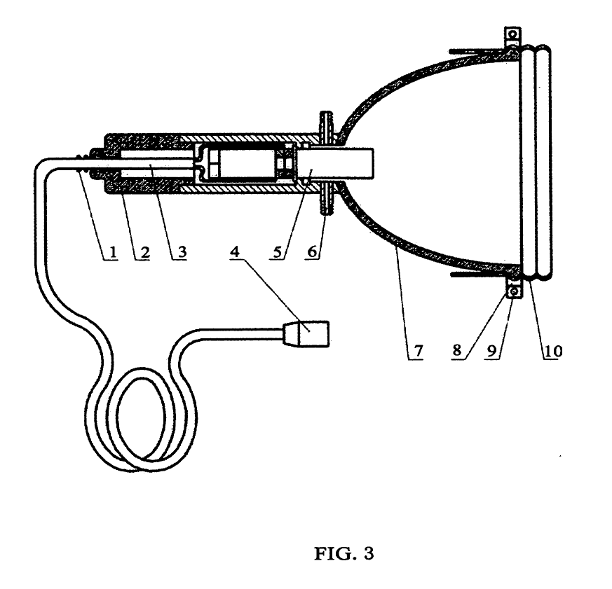

The invention provides a low-frequency focus ultrasonic treatment device comprising a spring 1, a hand grip 2, a cable 3, a plug 4, an ultrasound output 5, a tie-in for water infusion and gas exhaust 6, a half-ellipsoid reflector 7, a fixture 8, a screw 9, and a water sack 10, as exemplified in FIG. 3.

The low-frequency focus ultrasonic treatment device can be used as follows: formulating a microbubble agent and conveying it to a target region following a particular treatment requirement; providing energy for driving a ultrasonic transducer; conducting linkage; implementing tumor localization and treatment monitoring; transforming electrical energy into ultrasonic vibration; connecting the ultrasonic transducer and the ultrasonic therapy head; and applying a low-frequency focus ultrasonic treatment device (terminal conducting treatment); and so on.

The ultrasonic output 5 is used to transform electrical power into ultrasonic power. The front face (irradiation face) of the ultrasonic output 5 lies in a focus F1 of the half-ellipsoid reflector 7, and the tie-in 6 for water infusion and gas exhaust is in the lateral side of the connected region between the foreside of the hand grip 2 and the reflector 7. The inner portion of the tie-in is in communication with the inner portion of the half-ellipsoid reflector 7. At an opening on the forward end of the half-ellipsoid reflector is disposed the water sack 10. The half-ellipsoid reflector 7 and the water sack 10 are connected by the fixture 8 and the screw 9.

During treatment, degassed water is injected into the device through the tie-in 6 and the air is exhausted; electrical power is altered to ultrasonic power and output from the focus F1 of the reflector 7 through the ultrasonic output 5. The ultrasound is then gathered at the other focus F2 after reflection of the reflector 7, and then the ultrasonic biological effect is produced to treat the tumor.

What is claimed is

1. A low-frequency focus ultrasound-generating device for tumor treatment using ultrasound irradiation microbubble agent, characterized in that the device comprises: a hand grip, an ultrasound output, a half-ellipsoid reflector, and a water sack, wherein the half-ellipsoid reflector is disposed on the forward end of the hand grip; the rear part of the ultrasound output is set in the hand grip, while the front part of the ultrasound output is set in the half-ellipsoid reflector; on a lateral side of the ultrasound output in the front of the hand grip, there is a tie-in for water infusion and gas exhaust; the inner portion of the tie-in is in communication with the inner portion of the half-ellipsoid reflector; and at an opening on the forward end of the half-ellipsoid reflector is disposed the water sack.

2. The device as claimed in claim 1, characterized in that the front face of the ultrasonic output lies in a focus of the half-ellipsoid reflector.

3. The device as claimed in claim 1, characterized in that said water sack is fixed at the opening on the forward end of the half-ellipsoid reflector by the fixture.
